(19) United States
(12) Patent Application Publication
(10) Pub. No.: US 2005/0027539 A1
(43) Pub. Date: Feb. 3, 2005

(54) Media center controller system and method

(76) Inventors: Dean C. Weber, San Diego, CA (US); Laszio B. Betyar, Poway, CA (US); Kenneth R. Kubinak, Ramona, CA (US); James E. Hadzicki, San Diego, CA (US)

Correspondence Address: Wolff & King, PLLC, Suite 402, 2111 Eisenhower Ave., Alexandria, VA, 22314, US

(21) Appl. No.: 10/897,093

(22) Filed: Jul. 23, 2004

Related U.S. Application Data
(60) Provisional application No. 60/490,937, filed on Jul. 30, 2003.

(51) Int. Cl. A61N 1/18

(52) U.S. Cl. 704/275

(57) ABSTRACT

A system and methods for a media center controller. The system and methods include a computing device having a user dialog manager to process commands and input for controlling one or more controlled devices of the media center. The system and methods includes the capability to receive and respond to commands and input from a variety of sources, including spoken commands from a user, for remotely controlling one or more electronic devices and to perform, in response to the input received from the handheld device, speech recognition processing, voice over Internet Protocol communications, instant messaging, electronic mail messaging, or control of one or more controlled devices. The system and methods may also include a user interaction device capable of receiving spoken user input and transferring the spoken input to the computing device.

This application claims the benefit of U.S. Provisional Application No. 60/490,937, filed Jul. 30, 2003.

This disclosure contains information subject to copyright protection. The copyright owner has no objection to the facsimile reproduction by anyone of the patent disclosure or the patent as it appears in the U.S. Patent and Trademark Office files or records, but otherwise reserves all copyright rights whatsoever.

### BACKGROUND

1. Field of Invention

The present invention relates to media center control, and, more particularly, to media center control by a user.

2. General Background

Remotely controlled devices are commonplace today. Remote control devices typically have multiple buttons each one of which when actuated by a user may send a remote command to the remotely controlled device causing the controlled device to change its state of operation (e.g., change television channel or volume setting). Remote control devices may control a single device or multiple devices. A universal remote control has been developed that can control multiple different devices from different commercial manufacturers.

However, remote controls can be difficult to use in darkened rooms or under other conditions in which the button labels may be difficult to ascertain and, in any case, require the user to locate the button corresponding to the desired function. For example, users of a media center in a home or office may experience difficulty in attempting to control media devices or perform media related tasks using a remote control under conditions otherwise favorable to the media experience (e.g., seated or standing in a darkened room while directing attention to a display or screen). In some cases, voice command input may provide an easier user input mechanism.

### SUMMARY

Embodiments of the present invention may include a media center controller for controlling and providing user access to multiple devices and applications of a media center. Embodiments may also include systems and methods for transmitting and receiving speech commands from a user for remotely controlling one or more devices or applications. In at least one embodiment, a remote control device may be used as a voice command access point to control a variety of media related functions of a media center.

Embodiments may further include a media center controller that allows users to control various media center activities via manual devices, such as keypad or keyboard, or by voice command, which may include speaking naturally to their computers. Such activities may include playing music and DVDs, launching applications, dictating letters, browsing the Internet, using instant messaging, reading and sending electronic mail, and placing phone calls.

### BRIEF DESCRIPTION OF THE DRAWINGS

The invention claimed and/or described herein is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a system functional block diagram according to at least one embodiment;

FIG. 2 is a flow chart illustrating a method according to at least one embodiment;

FIG. 3 is a detailed functional block diagram of at least one embodiment of a media center controller according to the invention;

FIG. 4 is a detailed functional block diagram of a media center controller remote control device according to at least one embodiment;

FIG. 5 is a detailed functional block diagram of a media center controller computing device according to at least one embodiment; and

FIG. 6 is a logical control and data flow diagram depicting the transfer of information among various modules comprising the media center command processor according to at least one embodiment;

FIGS. 7a and 7b are a flow chart of a media center control method according to at least one embodiment;

FIG. 8 shows a top level menu interactive page according to at least one embodiment;

FIG. 9 shows a send voice recording interactive page according to at least one embodiment;

FIG. 10 shows a send e-mail interactive page according to at least one embodiment;

FIG. 11 shows a read e-mail interactive page according to at least one embodiment;

FIG. 12 shows a send text message interactive page according to at least one embodiment;

FIG. 13 shows a voice activated dialing interactive page according to at least one embodiment;

FIG. 14 shows a messenger interactive page according to at least one embodiment;

FIG. 15 shows a user account interactive page according to at least one embodiment;

FIG. 16 shows a user contacts interactive page according to at least one embodiment;

FIGS. 17a and 17b are a flowchart of a method voice over Internet Protocol (VoIP) or Personal Computer (PC)-to-PC applications in an embodiment; and

FIGS. 18a and 18b are a flowchart of a method 1800 for PC-to-phone applications in an embodiment.

### DETAILED DESCRIPTION

Described herein are a system and methods for a media center controller. The system and methods may include a computing device having a user dialog manager to process commands and input for controlling one or more controlled devices or applications. The system and methods may include the capability to receive and respond to commands and input from a variety of sources, including voice and manual entry commands and spoken commands from a user, for remotely controlling one or more electronic devices. In at least one embodiment, the system and methods may also include a user interaction device capable of receiving spoken user input and transferring the spoken input to the computing device. The user interaction device may be a handheld device.

Accordingly, embodiments of the present invention may include a system and method, interacting with a computer using a remote control device for controlling the computing device. Alternatively, other remote control devices may be used such as, for example, a Universal Remote Control device, which transmits utterances (i.e., spoken information) to a receiving computer device that may perform speech processing and natural language processing. The remote control device may include a microphone, and optionally a speaker, along with an optional microphone On/Off button. When actuated, the microphone On/Off button may mute the device(s) controlled by the remote control device, and begin its transmitting of the user's utterance to the receiving computing unit. When released, the microphone On/Off button may deactivate the microphone and un-mute the affected device(s) (such as, for example, television, stereo).

In at least one embodiment, the receiving computing unit may provide the audio transmission from the remote control device to a speech processing application and may transmit audio back to the remote control device for playback to the user using the speaker.

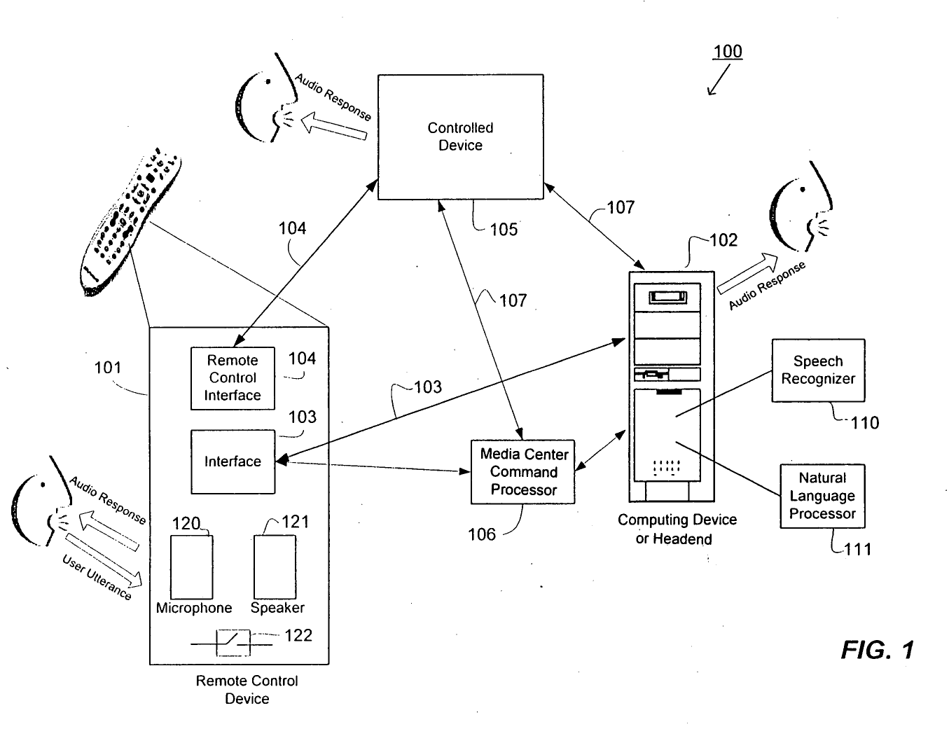

FIG. 1 is a system functional block diagram of at least one embodiment. Referring to FIG. 1, a system 100 may include a remote control device 101 which may be coupled to a computing device 102 using an interface 103. The remote control device 101 may also include a remote control interface 104 for transmitting commands to one or more controlled devices 105. In at least one embodiment, the remote control device may be a media center controller remote control unit. A media center command processor 106 may be coupled to or included with the computing device 102 and provided in communication with the remote control device 101 using the interface 103. Furthermore, in at least one embodiment the computing device 102 may be coupled to one or more controlled devices 105. In at least one embodiment, the computing device 102 may be a media center controller computing device.

In at least one embodiment, the computing device 102 may include a speech recognizer 110 and a natural language processor 111. The speech recognizer 110 and the natural language processor 111 may be implemented, for example, using a sequence of programmed instructions executed by the computing device 102. Alternatively, the speech recognizer 110 and the natural language processor 111 may comprise multiple portions of their respective applications, each of the portions executing on one or more of the computing device 102, and the media center command processor 106. In at least one embodiment, no training sequences are required by the speech recognizer 110.

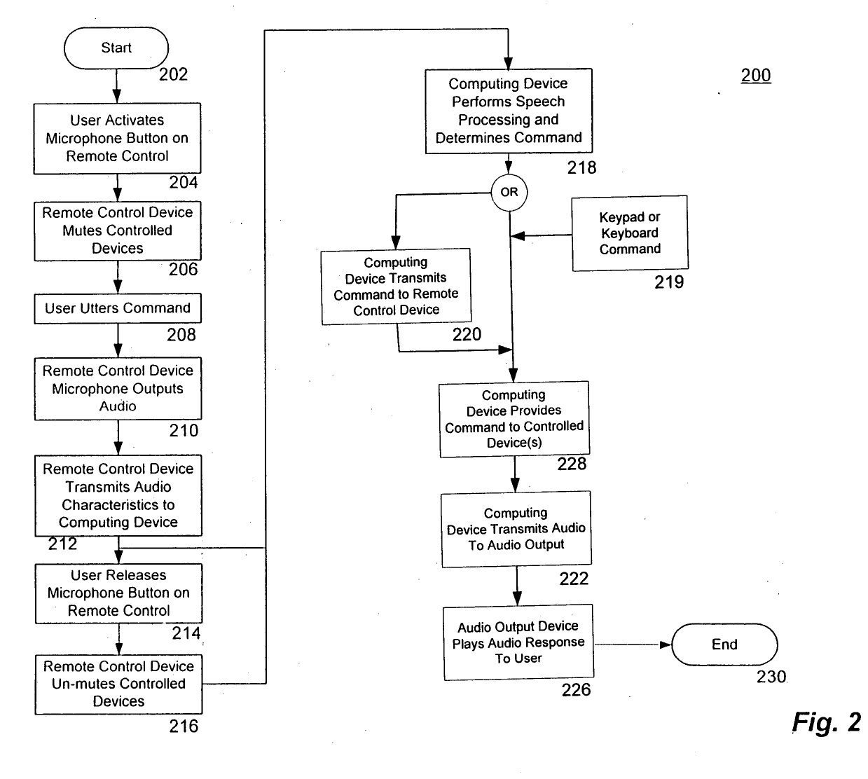

An example of a natural language processor is given in commonly assigned U.S. Pat. No. 6,434,524, entitled “OBJECT INTERACTIVE USER INTERFACE USING SPEECH RECOGNITION AND NATURAL LANGUAGE PROCESSING,” issued Aug. 13, 2002 (“the '524 patent”). In particular, the computing device 102 may be configured to include the natural language processor 111 as described with respect to the functional block diagram in FIG. 2 of the '524 patent and at col. 6, lines 13-67, which is hereby incorporated by reference as if set forth fully herein.

In an embodiment, the speech recognizer 110 may be configured to determine one or more remote control commands corresponding to the received audio signal. The speech recognizer 110 may include a speech processing capability that detects features of the audio signal sufficient to identify the corresponding remote commands or user requests or input. The mapping of the features to remote commands/requests may be maintained at the computing device 102 using, for example, non-volatile storage media such as a hard drive. Upon determining the remote command(s) or input, the computing device 102 sends the corresponding response(s) to the remote control device 101 using the interface 103.

In an embodiment, the audio signal may be input to the natural language processor 111 for extraction of the relevant portions of the audio signal required for the speech recognizer 110 to determine the associated command or input. The natural language processor 111 may receive the audio signal prior to the speech recognizer 110, at the same time as the speech recognizer 110, or only if the speech recognizer 110 first fails to confidently determine the corresponding remote command. Upon receiving the remote command or interpreted information from the computing device 102, the remote control device 101 may output the remote command to the affected controlled device(s) 105 using the remote control interface 104.

In an embodiment, one or both of the speech recognizer 110 and the natural language processor 111 may be implemented in the media center command processor 106 which is coupled to or included with the computing device 102. In particular, the media center command processor 106 may include hardware and software components to perform the speech analysis described above, thereby reducing the processing load and processing bandwidth requirements for the computing device 102. The media center command processor 106 may be operably coupled to the computing device 102 using a variety of known interfacing mechanisms (e.g., USB, Ethernet, RS-232, parallel port, IEEE 802.11). In at least one embodiment, the media center command processor 106 may be coupled to the controlled device(s) 105 using a network 107. The media center command processor 106 may be a set top box. Alternatively, the media center command processor 106 may be implemented as one or more internal circuit board assemblies, software or a sequence of programmed instructions, or a combination thereof, of the computing device 102. Alternatively, the media center command processor 106 may be implemented using hardware and software in the remote control device 101 or one or more of the controlled devices 105.

In an embodiment, the computing device 102 and media center command processor 106 may be implemented using one or more computing platforms of a headend system for cable or satellite television or media signal distribution. In particular, the computing device 102 may be provided using one or more servers, which may be PC-based servers, at the headend. In these embodiments, the media center command processor 106 may be implemented as one or more internal circuit board assemblies, software or a sequence of programmed instructions, or a combination thereof, of the headend. The remote control device 101 may output remote control signals (either keypad command or voice input) to the headend computing device 102 via the interface 103. In these embodiments, the interface 103 may be a satellite channel or a cable channel for communications in the direction from the user to the headend. A Cable Television (CATV) converter box may be provided for transmitting information back to the CATV service provider or headend from the remote control device 101.

In at least one embodiment, the remote control device 101 may include buttons which, when actuated by a user, cause the transmission of remote commands or status inquiries to the controlled device(s) 105 using the remote control interface 104. Furthermore, the remote control device 101 may be capable of controlling a single device, multiple devices, or may be a Universal Remote Control device capable of controlling multiple controlled devices 105 provided by different manufacturers. Alternatively, the remote control device 101 may be a Bluetooth™ capable headset. The remote control device 101 may allow user selection of a particular controlled device 105 to be controlled using the remote control device 101. In an embodiment, the remote control device 101 may include at least one processor such as, but not limited to, a microcontroller implemented using an integrated circuit. In some embodiments, the remote control device 101 may simultaneously send or broadcast information to more than one controlled device 105. In an embodiment, the remote control device 101 may include a microphone 120, a speaker 121, and a switch 122 operable to actuate the microphone and transmit information using the interfaces 103 and 104.

In at least one embodiment, actuation of the switch 122 may cause information to be sent to one or more controlled devices 105 using the remote interface 104 that causes the audio output of those devices 105 to be muted while the switch is actuated. Alternatively, the information or command that causes the muting may be sent from the media center command processor 106 or the computing device 102 directly to the controlled device 105. While the switch 122 is actuated, the interface 103 transmits audio signal of the audio received from the microphone 120 (spoken by a user, for example) to the computing device 102. The audio signal may be encoded or compressed using a variety of compression algorithms (e.g., coder-decoder (CODEC), vocoding) to reduce the amount of information transferred using the interface 103, and its attendant bandwidth and data rate requirements. In an embodiment, the remote control device 101 may be configured to extract particular features from the audio received from the microphone 120.

In at least one embodiment, the remote control device 101 may include a pushbutton by which a user may actuate and release the switch 122. Alternatively, the switch 122 may be voice activated. Upon the user releasing the switch 122, the remote control device 101 may turn deactivate the microphone 120, cease sending information to the computing device 102 via interface 103, and send an “un-mute” command via remote control interface 104 or interface 107 to the controlled devices 105. This approach reduces the power consumed by the remote control device 101. Alternatively, the mute and un-mute signals may be sent by the computing device 102, in which case the computing device 102 may also include a remote control interface 104; or, the mute and un-mute signals may be sent by the media center command processor 106 via the interface 107, or by the remote interface 104 (if present at the media center command processor 106).

In addition, the remote control device 101 may include one or more programmable switches and a coder that transmits codes over the remote control interface 104 based on the switch settings as determined by a switch state to code mapping maintained by the remote control device 101. In an embodiment the switches may be programmed by a user interacting with a user interface of the remote control device 101. Alternatively, the switches may be programmed by the computing device 102 using the interface 103. Alternatively, the switch state to code mapping is maintained by the computing device 102 and downloaded to the remote control device 101 using the interface 103.

In an embodiment, the computing device 102 may be implemented using a personal computer configured to execute applications compatible with the Windows™ operating system available from Microsoft Corporation of Redmond, Wash. For example, in at least one embodiment, the computing device 102 may execute the Microsoft™ Windows Media Center™ operating system. Other embodiments are possible, including other operating systems and computing platforms. For example, the computing device may be implemented using a game device console (e.g., X-Box™, Sony Playstation™ or Playstation2™, or GameCube™), a television set top box, a digital video recorder (e.g., TiVo™, Replay TV™), a home theater sound processor, or other processing device. In at least one embodiment, all or a portion of the systems and methods described herein may be implemented as a sequence of programmed instructions executing on the computing device 102 along with and in cooperation with other processors or computing platforms. In at least one embodiment, the computing device 102 may include a sound card/Universal Serial Bus (USB) port for input of audio signal.

In an embodiment, the computing device 102 may include an audio response capability. In particular, upon receiving the audio signal from the remote control device 101, the computing device 102 may provide an audio response to the remote control device 101 using the network 103. Upon receiving the audio response information from the computing device 102, the remote control device 101 may output the audio response to the user using the speaker 121. Accordingly, the audio response information may be synthesized speech provided by the computing device 102. Alternatively, the audio response information may be stored actual speech information from a human voice, or fragments thereof, or may be generated as required using a speech synthesis application. In an embodiment, the audio response information may produce audio confirming to the user that the operation requested in the audio signal (e.g., spoken request from the user) have been accomplished. For example, if the user utters “TV channel 27,” upon the system changing the television controlled device to channel 27 as described herein, an audio response stating “TV Channel 27” may be played to the user over speaker 121. Other messages are possible, such as “Television 1 changed to channel 27,” etc.

Alternatively, these audio response functions may be performed by the computing device 102 without involving the remote control device 101, by using, for example, the interface 107. In such embodiments, the audio response may be played from a speaker on the computing device 102 (the computing device 102 having a sound card) or from a speaker of one or more of the controlled devices 105. Alternatively, the media center command processor 106 may provide some or all of these audio response functions, or may share them with the computing device 102.

Controlled devices 105 may include electronic devices produced by different manufacturers such as, for example, but not limited to, televisions, stereos, video cassette recorders (VCRs), Compact Disc (CD) players/recorders, Digital Video Disc (DVD) players/recorders, TiVo™ units, satellite receivers, cable boxes, television set-top boxes, the Internet and devices provided in communication with the Internet, tuners, and receivers. The remote control interface 104 may include, for example, an InfraRed (IR) wireless transceiver for transmission, and possibly reception, of command and status information to and from the controlled devices 105, as is commonly practiced. However, the remote control interface 104 may be implemented according to a variety of techniques in addition to IR including, without limitation, wireline connection, a Radio Frequency (RF) interface, telephone wiring carried signals, BlueTooth™, Firewire™, 802.11 standards, cordless telephone, or wireless telephone or cellular or digital over-the-air interfaces.

Alternatively, the computing device 102 may be configured as an Interactive Voice Response (IVR) system. In particular, the computing device may be configured to support a limited set of IVR command-response pairs such as, for example, command-responses that accomplish pattern matching for the received audio signal without semantic recovery.

The interfaces 103 and 107 may be an electronic network capable of conveying information such as, for example, an RF network. Examples of such an RF network include Frequency Modulation (FM), IEEE 802.11 standard and variations, IR, Firewire™, and Bluetooth™. Further, the interface 103 may be a satellite communication channel or a Cable Television (CATV) channel. Other networks are possible.

The remote control device 101 may include navigation keys 301, a numeric and text entry keypad 302, a microphone 120, a speaker 121, a mute button or switch 122, an interface 103, and a remote control interface 104. The interface 103 may further include an audio receiver 303, an audio transmitter 304, and a function key transmitter 305.

In another embodiment, the telephone customer premises equipment (CPE) may be used to obtain and process a user's audio utterances for remote control. In particular, the remote control device 101 may be implemented using a telephone handset (which may be a wireline or a cordless or cellular/mobile handset or headset) having the speech processing capabilities described herein. Audio signal may be transmitted from the telephone handset to the computing device 102 using the existing household telephone wiring. The handset microphone and speaker may be used for obtaining the user's utterances and for playback of the audio response, respectively. The remote command information received from the computing device 102 may be transmitted by the handset to the controlled device(s) 105 using the interface 103 included in the handset for this purpose. In addition, the computing device 102 may output audio queries to the user via the handset speaker (e.g., “What do you want to do?”).

FIG. 2 is a flow chart of a method 200 according to at least one embodiment. Referring to FIG. 2, the method 200 may commence at 202. Control may then proceed to 204 at which the user activates the microphone button on the remote control device. In response, at 206, the remote control unit may mute the controlled device(s). Upon the user uttering a command at 208, the remote control device microphone may output (for example, by streaming) the audio uttered by the user at 210 and transmit the audio signal to the computing device at 212.

Upon the user releasing the microphone button at 214, the remote control device (or the computing device or media center command processor) may unmute the controlled device(s) at 216.

Upon receiving the audio signal, the computing device may perform speech processing as described above to determine the associated remote command(s) at 218. The computing device may then transmit the corresponding response (which may be a device command) to the remote control device at 220. Or, if the input is a non-spoken input, a keypad or keyboard input may be received at 219. Control may then proceed to 228, at which the computing device provides the command to the controlled device(s).

The computing device may also transmit an audio response to an audio output device at 222. Upon receiving the audio response, the audio output device may play the audio response to the user using a speaker at 226. Alternatively, the computing device may output the audio response directly to the controlled device to play over a speaker of the controlled device. At 230, the method may end.

In at least one embodiment, the above described system and method may be used for media center control. A media center may be any system that includes a processor configured to provide control and use of multiple media devices or capabilities. Examples of such media devices include, but are not limited to, Television (TV), cable TV, direct broadcast satellite, stereo, Video Cassette Recorder (VCR), Digital Video Disc (DVD), Compact Disc (CD), Tivo™ recorder, and World Wide Web (WWW) browser, electronic mail client, telephone, voicemail. One or more of these media devices may be implemented using application software programmed instructions executing on a personal computer or computer platform.

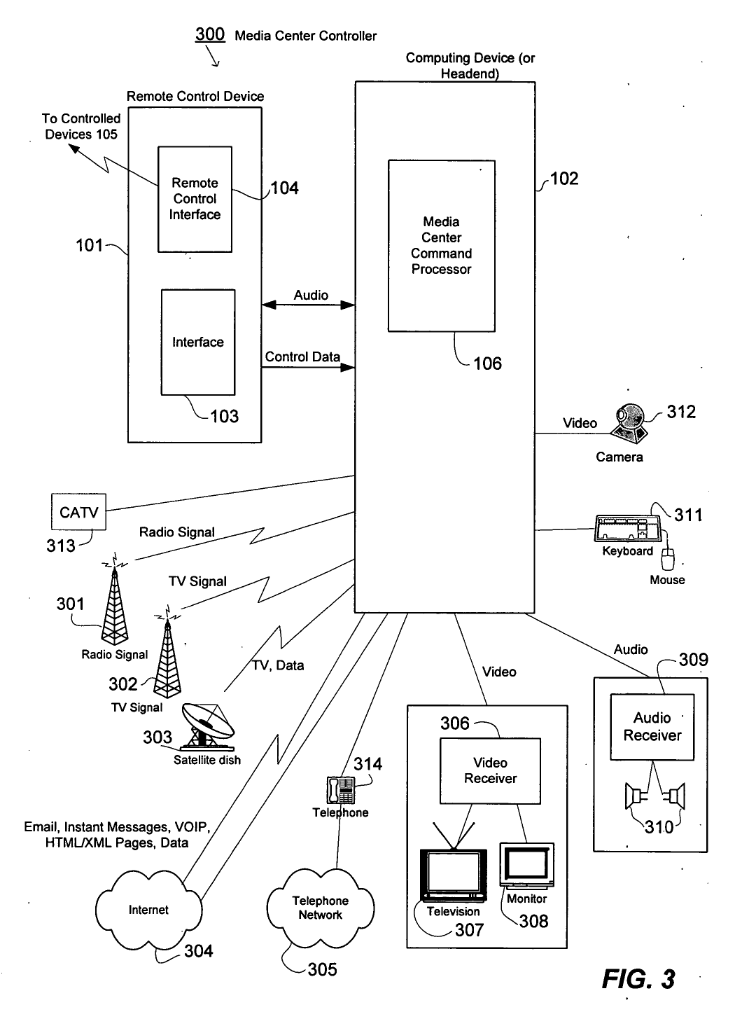

FIG. 3 is a detailed functional block diagram of a media center controller 300 according to at least one embodiment. Referring to FIG. 3, the media center controller 300 may include a computing device 102, which may be a media center controller computing device. In at least one embodiment, the computing device 102 may be coupled to a remote control device 101, which may be a media center controller remote control device, for receiving and transmitting audio information and for receiving control data from the remote control device 101. As shown in FIG. 3, the computing device 102 may include the media center command processor 106. In at least one embodiment, the media command processor 106 may include a speech transceiver capability. Further, the computing device 102 for media center controller 300 may be operably coupled to a variety of media devices as described above. As shown in FIG. 3, the media center controller computing device 102 may be operably coupled to, for example, but not limited to, a radio signal source 301 for receiving radio broadcast signals, a Television (TV) signal source 302 for receiving TV broadcast signals, a satellite signal source 303 for receiving satellite transmitted TV and data signals, including direct broadcast satellite TV and data signals, a CATV converter box 313 for communication to and from a CATV headend, and to a private or public packet switched network 304 such as, for example, the Internet, for receiving and transmitting a variety of packet based information to other PCs or other communications devices. Packet based information transferred by the computing device 102 includes, but is not limited to, electronic mail (email) messages in accordance with SMTP, Instant Messages (IM), Voice-Over-Internet-Protocol (VoIP) information, HTML and XML formatted pages such as, for example, WWW pages, and other packet or IP based data.

Further media devices to which the media center controller computing device 102 may be operably coupled to include, for example, but are not limited to, a wireline or cordless access telephone network 305 such as the Public Switched Telephone Network (PSTN), and wireless or cellular telephone systems. In such embodiments, the computing device 315 may be coupled to a telephone handset 315, which may be a cordless or wireless handset.

In addition, in an embodiment, the computing device 102 may be optically or electronically coupled to a keyboard and mouse 311 for receiving command and data input, as well as to a camera 312 for receiving video input. The computing device 102 may also be coupled to a variety of known video devices, optionally using a video receiver 306, for output of video or image information to a television 307, computer monitor 308, or other display device. The computing device 102 may also be coupled to a variety of known audio devices, optionally using an audio receiver 309, for output of audio information to one or more speakers 310. Furthermore, the media center controller 300 may include an audio file/track player to play audio files requested by the user; and an audio/visual player to play audio/visual files or tracks requested by the user.

With respect to FIG. 3, in an embodiment, the computing device 102 and media center command processor 106 may be implemented using one or more computing platforms of a headend system for cable or satellite television or media signal distribution. In particular, the computing device 102 may be provided using one or more servers, which may be PC-based servers, at the headend. In these embodiments, the media center command processor 106 may be implemented as one or more internal circuit board assemblies, software or a sequence of programmed instructions, or a combination thereof, of the headend. The remote control device 101 may output remote control signals (either keypad command or voice input) to the headend computing device 102 via the interface 103. In these embodiments, the interface 103 may be a satellite channel or a cable channel for communications in the direction from the user to the headend. Further, the media center controller 300 may include a CATV converter box for transmitting information back to the CATV service provider or headend from the remote control device 101.

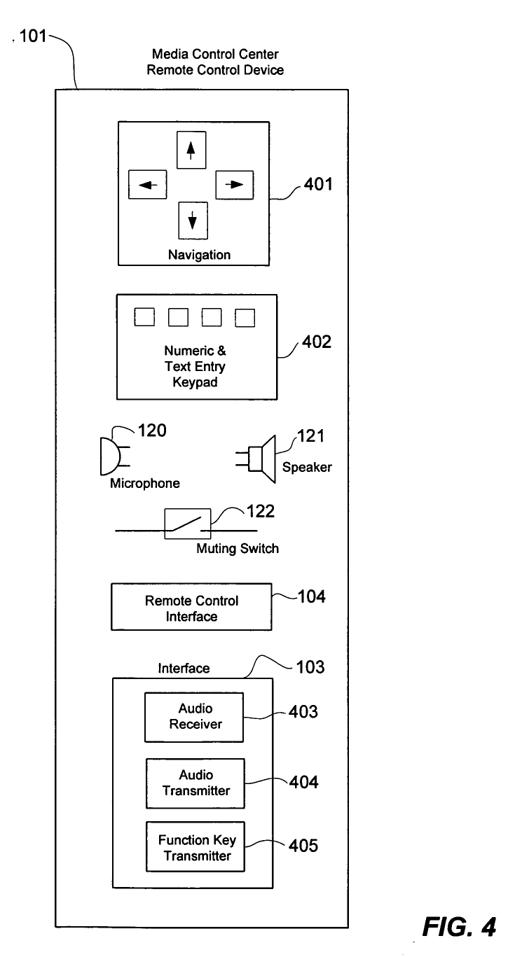

FIG. 4 is a detailed functional block diagram of a media center controller remote control device 101 according to at least one embodiment. Referring to FIG. 4, the remote control device 101 may include navigation buttons 401 operable to allow a user to input directional commands relative to a cursor position or to scroll among items for selection using a display, a numeric and text entry keypad 402 operable to allow a user to input numeric and text information, the microphone 120 for receiving user voice utterances, the speaker 121 for providing audio output to a user, the activation/mute switch 122 for muting controlled devices, the remote control interface 104 for sending information to controlled devices, and the interface 103 for transferring audio to and from and control data to the computing device 102. The remote control device 101 may further include a ‘clear’ button and an ‘enter’ button. In an embodiment, the interface 103 may include an audio receiver portion 403, an audio transmitter portion 404, and a function key transmitter portion 405, for transferring this respective information to the computing device 102.

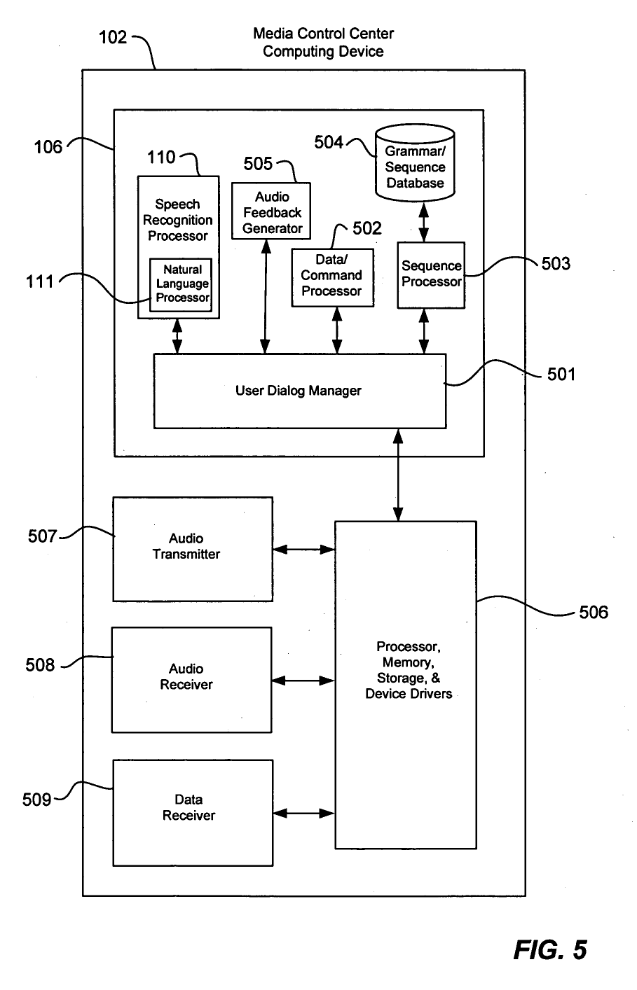

FIG. 5 is a detailed functional block diagram of a media center controller computing device 102 according to at least one embodiment. Referring to FIG. 5, the computing device 102 may include the media center command processor 106. The computing device 102 may also include standard computer components 506 such as, but not limited to, a processor, memory, storage, and device drivers. In an embodiment, the computing device 102 may be a Microsoft Windows™ compatible PC provided by a variety of manufacturers such as the Dell Corporation of Austin, Tex. The computing device 102 may also include an audio transmitter 507 for transferring synthesized speech and other audio output to the remote control device 101, an audio receiver, or other controlled device for output to a listening user. The computing device 102 may also include an audio receiver 508 for receiving audio information from the remote control device 101 or a microphone. Further, the computing device 102 may include a data receiver 509 for receiving function key, keypad, or navigation key information from the remote control device 101, and for receiving keyboard or mouse input, and for receiving packet based information. Other types of received data are possible.

In at least one embodiment, the media center command processor 106 may include the speech recognition processor 110, an audio feedback generator 505 that may include a speech synthesizer, a data/command processor 502, a sequence processor 503, and a user dialog manager 501. The speech recognition processor 110 may further include the natural language processor 111. In an embodiment, each of these items comprising the media center command processor 106 may be implemented using a sequence of programmed instructions which, when executed by a processor such as the processor 506 of the computing device 102, causes the computing device 102 to perform the operations specified. Alternatively, the media center command processor 106 may include one or more hardware items, such as a Digital Signal Processor (DSP), to enhance the execution speed and efficiency of the voice processing applications described herein. In an embodiment, the speech recognition processor 110 may receive the audio signal and convert or interpret it to one or more particular commands or to input data for further processing. In addition to command grammar processing, natural language processing may also be used for voice command interpretation. Further details regarding the interaction between the user dialog manager 501 and the speech recognition processor 110 for natural language processing are set forth in commonly assigned U.S. Pat. No. 6,532,444, entitled “USING SPEECH RECOGNITION AND NATURAL LANGUAGE PROCESSING,” issued Mar. 11, 2003 (“the '444 patent”), which is hereby incorporated by reference as if set forth fully herein. In particular, the computing device 102 may be configured to include the natural language processor 111 and speech recognition processor 110 as described with respect to the functional block diagram in FIG. 2 of the '444 patent.

In that regard, the speech recognition processor 110 may include a natural language processor 111 as described herein to assist in decoding and parsing the received audio signal. For example, the natural language processor 111 may be used to identify or interpret an ambiguous audio signal resulting from unfamiliar speech phraseology, cadence, words, etc. The speech recognition processor 110 and the natural language processor 111 may obtain expected speech characteristics for comparison from the grammar/sequence database 504. The audio feedback generator 505 may be configured to convert stored information to a synthesized spoken word recognizable by a human listener, or to provide a pre-stored audio file for playback. The data/command processor 502 may be configured to receive and process non-spoken information, such as information received via keyboard, remote 101 keypad, email, or VoIP, for example. The sequence processor 503 may be configured to retrieve and executed a predefined spoken script or a predefined sequence of steps for eliciting information from a user according to a hierarchy of different command categories. The sequence processor 503 may also validate the input received as being at the proper or expected step of a sequence or scenario. The sequence processor 503 may obtain the sequence information from the grammar/sequence database 504. In addition, the sequence processor 503 may determine an appropriate response for output to the user based on the received user input. In making this determination, the sequence processor 503 may use or consult a sequence or set of steps associated with the input and the context of the task requested or being performed by the user.

The user dialog manager 501 may provide management for functions such as, but not limited to: determining whether input received from an application includes an audio signal for speech recognition or is command/data input for command interpretation; requesting command validation and response identification from the sequence processor; outputting audio or display based responses to the user; requesting text to speech conversion or speech synthesis; requesting audio and/or visual output processing; and calling operating system functions and other applications as required to interact with the user.

In at least one embodiment, the media center command processor 106 may further comprise a grammar/sequence database 504. The grammar/sequence database 504 may include predefined sequences of information, each of which may be used by the sequence processor 503 to output information or responses to a user designed to elicit information from the user necessary to perform a media related function in a contextually proper manner. Further, the grammar/sequence database 504 may include state information to specify the valid states of a task, as well as the permissible state transitions.

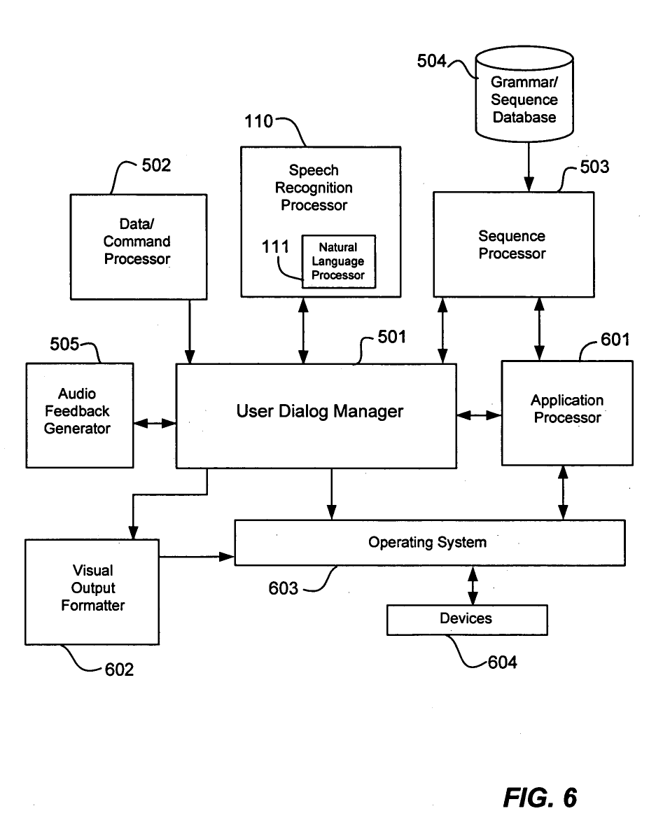

FIG. 6 is a logical control and data flow diagram depicting the transfer of information among various modules of the media center command processor 106 according to at least one embodiment. Referring to FIG. 6, the user dialog manager 501 may receive user input from a variety of input devices via an application processor 601. The application processor 601 may be configured to receive input from a user via spoken information such as, for example, audio signals received from the remote control device 101, as well as to receive non-spoken information, such as information received via keyboard manual entry, remote 101 keypad, or Voice Over Internet Protocol (VOIP), for example. The user dialog manager 501 may transfer the audio signal to the speech recognition processor 110 for interpretation of the received audio signal into command or data information. The user dialog manager 501 may transfer command information to the data/command processor 502 for further processing such as, for example, validation of the received input in the context of the requested task or task in process.

The user dialog manager 501 may also request the sequence processor 503 to validate that the received input is within an acceptable range and is received in the proper or expected sequence for an associated task. If the input is valid and in-sequence, the sequence processor 503 may identify to the user dialog manager 501 an appropriate response to be output to the user. Based on this response information, the user dialog manager 501 may request the audio feedback generator 505 to prepare an audio response to be output to the user, or may play a pre-recorded prompt. The user dialog manager 501 may also request a visual output formatter 602 to prepare a visual response to be output to the user. The user dialog manager 501, the visual output formatter 602, and the application processor 601 may output the user response to an operating system 603 of the computing device 102 as well as to applications or device drivers for a variety of output devices 604 for output to the user, such that the user dialog manager 501 is logically connected through operating system services to input/output devices.

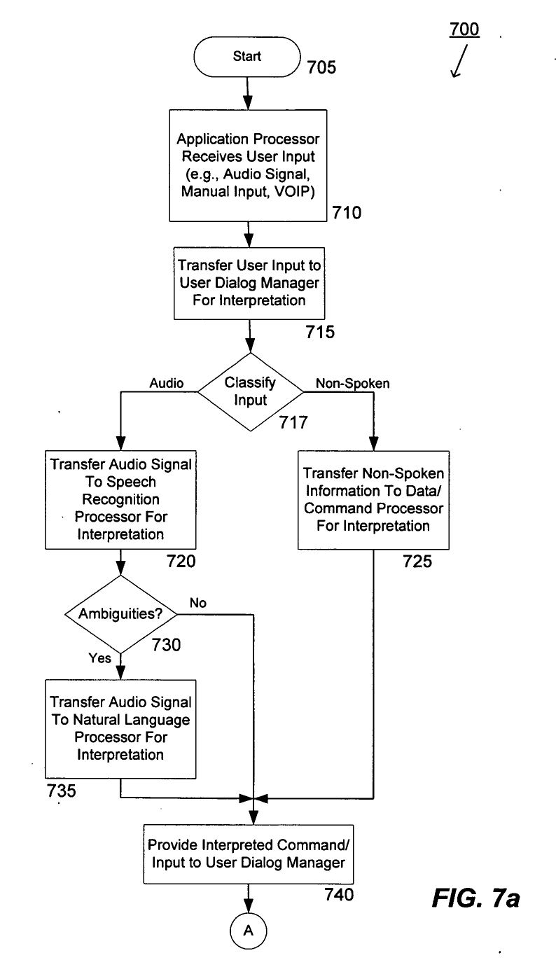

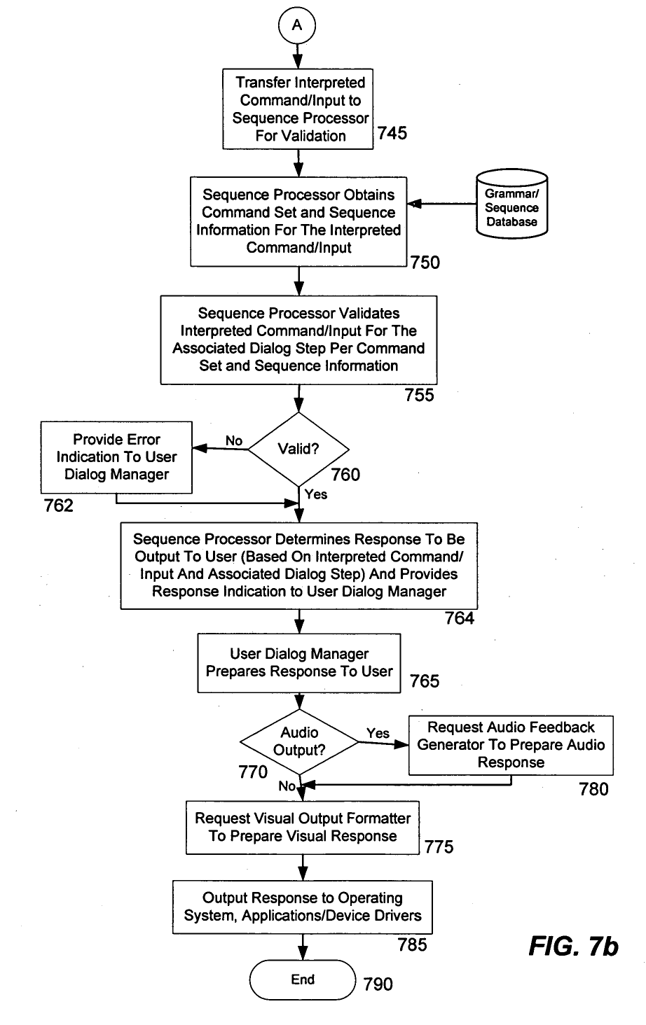

FIGS. 7a and 7b illustrate a flow chart of a media center control method 700 according to at least one embodiment. Referring to FIG. 7a, a method 700 may commence at 705. Control may then proceed to 710, at which user input is received by an application or an application processor. The input may be received from a user via spoken information such as, for example, audio signals received from the remote control device 101, but may also include non-spoken information, such as information received via keyboard manual entry, remote 101 keypad, or VOIP, for example.

Control may then proceed to 715, at which the application processor may transfer the user input (e.g., audio signal, commands, data) to the user dialog manager for interpretation. Control may then proceed to 717, at which the user dialog manager may classify the input as audio or non-spoken input. At 720, the user dialog manager may then transfer the audio signal to the speech recognition processor for interpretation of the audio signal into command or data information. At 725, the user dialog manager may transfer non-spoken information to the data/command processor for further processing such as, for example, validation of the received input in the context of the requested task or task in process.

If at 730 the speech recognition processor determines that the received audio signal includes ambiguities such as extraneous information, noise, or otherwise are not readily susceptible of interpretation, then control may proceed to 735 at which natural language processing may be performed. The natural language processing may provide for additional interpretation of the audio signal for determining the requested command, operation, or input.

Control may then proceed to 740, at which the speech recognition processor or data/command processor provide an indication of the interpreted command(s) or input to the user dialog manager. Referring to FIG. 7b, control may then proceed to 745, at which the user dialog manager may transfer the interpreted command(s) or input to the sequence processor for validation. At 750, the sequence processor may obtain command set and sequence information associated with the interpreted command(s) or input from the grammar/sequence database. Control may then proceed to 755, at which the sequence processor may validate that the interpreted command or input is within an acceptable range and is received in the proper or expected sequence or dialog step for an associated task as specified in a predefined state table contained in the grammar/sequence database. If at 760 the sequence processor determines that the interpreted command or input is valid, then control may proceed to 764; otherwise, control proceeds to 762 at which the sequence processor provides an error indication to the user dialog manager indicating command/input validation failure.

At 764, if the input is valid and in-sequence, the sequence processor may identify to the user dialog manager an appropriate response to be output to the user. Control may then proceed to 765, at which, based on this response information, the user dialog manager may prepare a response to the user. Control may then proceed to 770, at which the user dialog manager may determine if an audio output response is to be provided. If so, control may then proceed to 780 at which the user dialog manager requests the audio feedback generator to prepare an audio response to be output to the user, or plays a pre-recorded audio file. In either case, at 775 the user dialog manager may request the visual output formatter to prepare a visual response to be output to the user. Control may then proceed to 785, at which the user dialog manager, the visual output formatter, and the application processor may output the user response to an operating system of the computing device as well as to applications or device drivers for a variety of output devices for output to the user. At 790, a method may end.

In at least one embodiment, the media center controller 300 may be used for control of and interaction with a variety of media devices and functions. For example, the media center controller may allow a user to command a platform (device or computer) to implement capabilities such as, but not limited to: making audio phone calls; making video phone calls; instant messaging; video messaging; sending voice recordings; reading e-mail; sending e-mail; sending text messages; managing user contacts; accessing voice mail; calendar management; playing music; playing movies; playing the radio; playing TV programs; recording TV programs; browsing the Internet; dictating documents; entering dates into a personal calendar application and having the system provide alerts for upcoming scheduled meetings and events; and launching applications. In an embodiment, the mechanism for interaction with the computer system is accomplished through either a) a remote control device, b) microphone input, c) keyboard and mouse, or d) touch-screen. With respect to the remote control device, it may be a multi-mode input device that has a keypad for manual entry of commands transmitted to the system, as well as a microphone embedded in the remote control, allowing the user to provide spoken commands to the system. As discussed herein, in at least one embodiment, the media center controller 300 may include a natural language interface allows users to speak freely and naturally. However, manual key, touchscreen and keyboard/mouse interface may also be provided as an option to speech. In an embodiment, the media center controller 300 may provide a mechanism, such as a logon authentication process using an interactive page, to identify the current user and allow or deny access to the system.

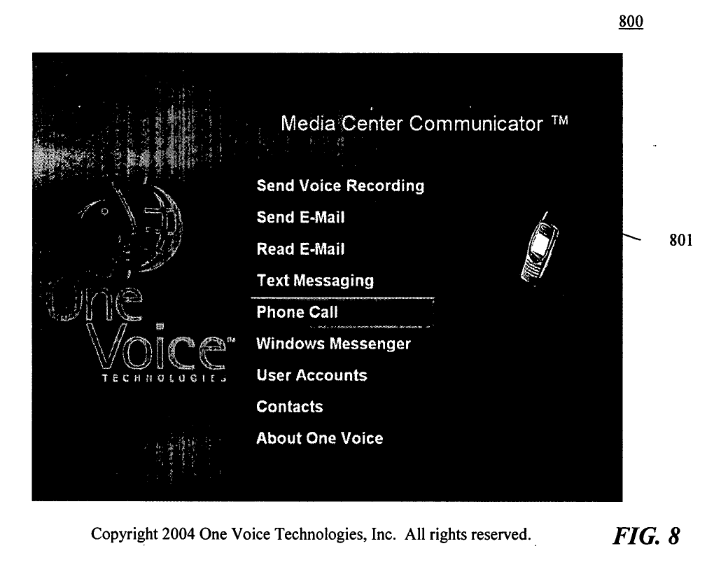

FIG. 8 shows a top level menu interactive page 800 according to at least one embodiment. Referring to FIG. 8, the top level menu interactive page 800 may include several media function selection buttons 801. Upon user selection of a particular media function selection button 801, a request to execute the associated media function may be received by the application processor 601. The application processor 601 may forward the request to the user dialog manager 501 for processing as described with respect to FIGS. 7a and 7b herein.

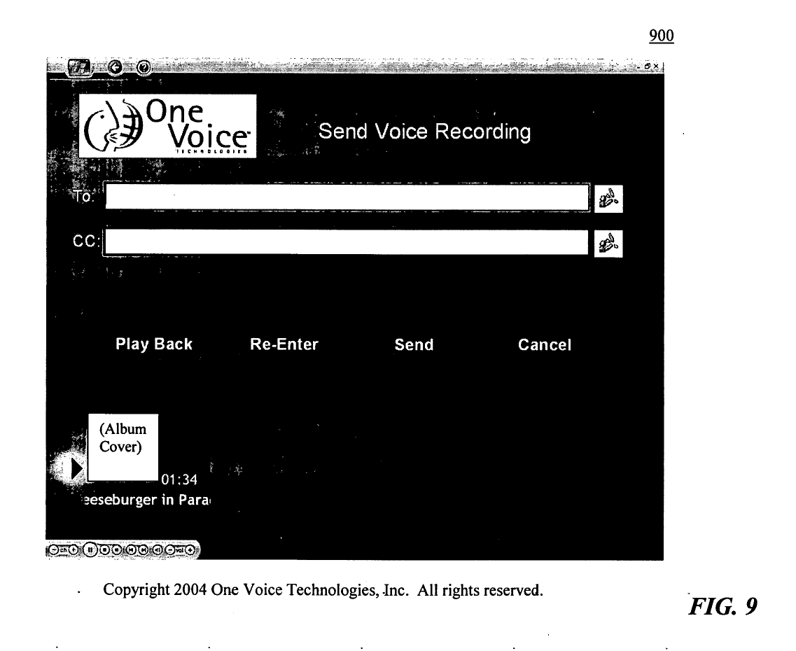

FIG. 9 shows a send voice recording interactive page 900 according to at least one embodiment. Referring to FIG. 9, the send voice recording interactive page 900 may provide an interface by which a user may compose and send a recorded voice message for a wireless device. Using this feature, the user can record a voice message to a recipient, such as a contact, and then send the recorded voice message to the recipient. The media center controller 300 may record the voice message as a .wav file, for example. The recipient listener will hear exactly what the recording user says, so misinterpretation can be avoided. In an embodiment, the recorded voice message may be delivered to the recipient's inbox as an e-mail. When the recipient opens the e-mail message, they will hear the .wav file play your message.

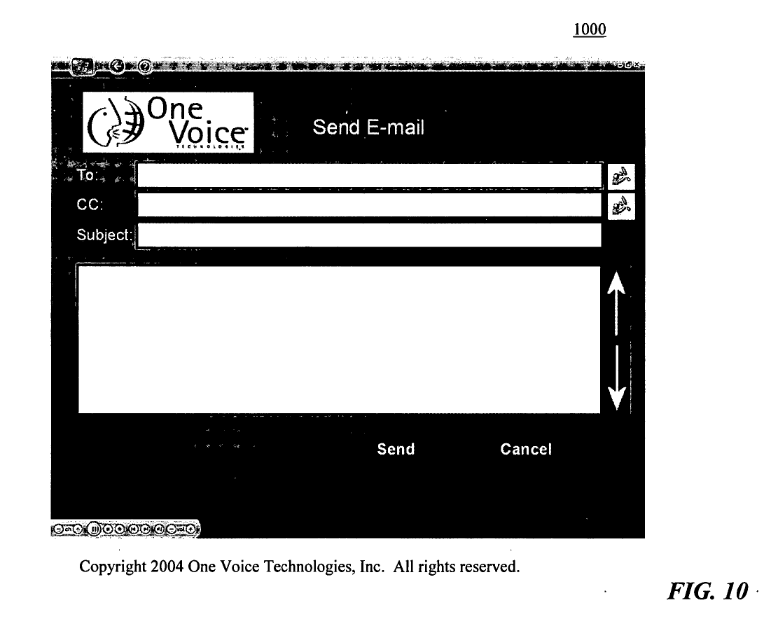

FIG. 10 shows a send e-mail interactive page 1000 according to at least one embodiment. Referring to FIG. 10, the send e-mail interactive page 1000 may provide an interface by which a user may compose and send an e-mail message for a wireless device. Using this feature, the user may speak his message into the wireless device and his voice is converted to text as discussed herein. The e-mail message may be sent to the recipient using a network, and will appear in the recipient's inbox as if it was written on a computer. In at least one embodiment, the send e-mail feature requires no keypad tapping to create. While the user dictates the message, he may be provided the option to edit, add more, or send.

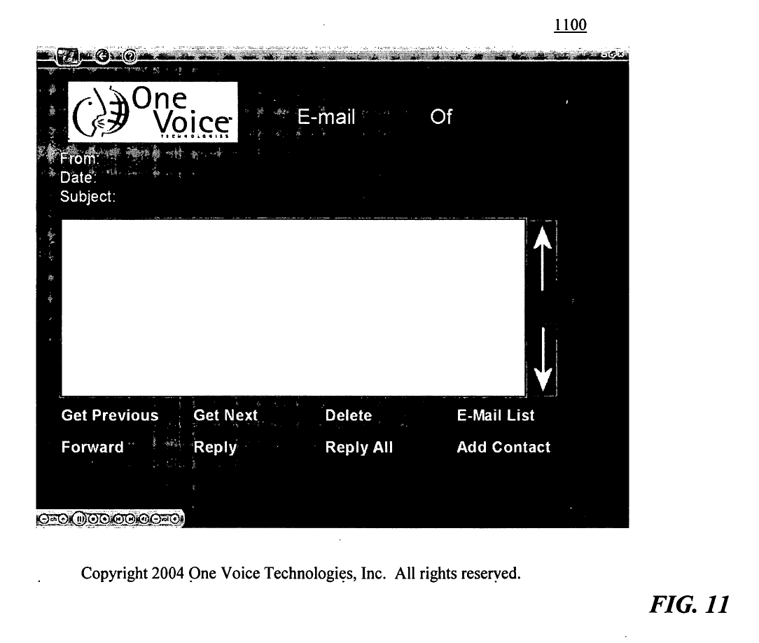

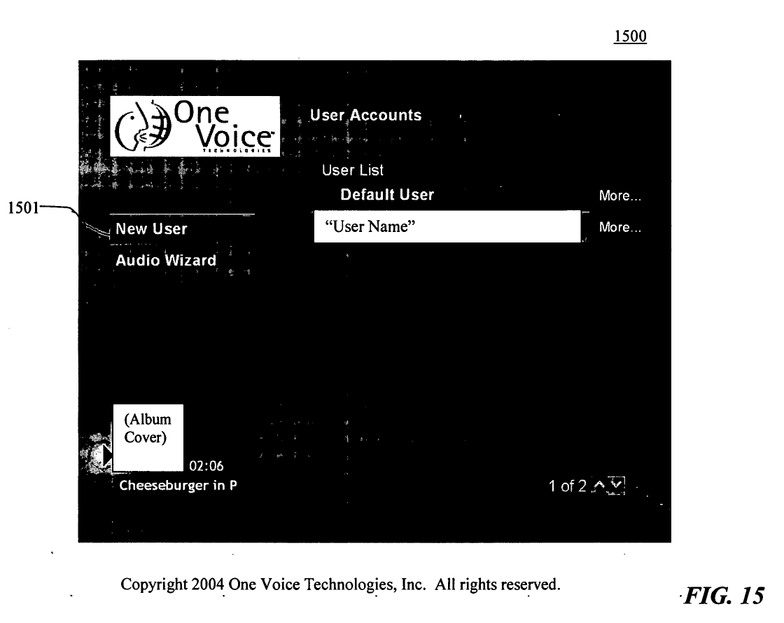

FIG. 11 shows a read e-mail interactive page 1100 according to at least one embodiment. Referring to FIG. 11, the read e-mail interactive page 1100 may provide an interface by which a user may read an e-mail message. In at least one embodiment, users may access their corporate or personal e-mail account via the Media center controller. In order to use the E-mail Read feature, a POP3, IMAP, or corporate e-mail account may be required. To use this feature, a user first enters her e-mail server name, account name, and password into the user profile portion (see FIG. 15) of the read e-mail interactive page 1100. Next, the entered information may be stored by the computing device of the media center controller. Thereafter, when the user calls in, she will be able to check her e-mail by saying “Read E-mail.” In an embodiment, users may have the option to reply to, forward, delete, and skip e-mails.

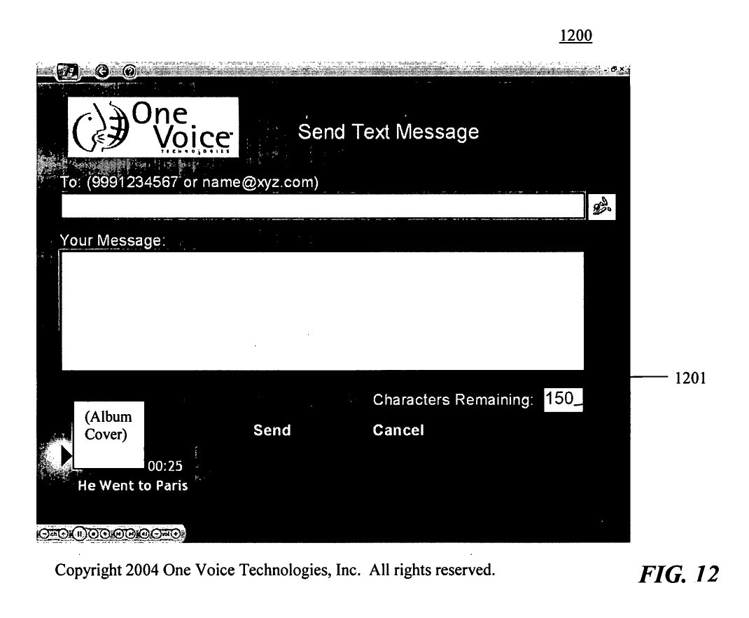

FIG. 12 shows a send text message interactive page 1200 according to at least one embodiment. Referring to FIG. 12, the send text message interactive page 1200 may provide an interface by which a user may send a text message. Text messaging is a way to send short messages from wireless device to a wireless phone. In an embodiment, users may send text messages such as, for example, SMS messages, to anyone with a messaging-capable phone. In at least one embodiment, the send text message interactive page 1200 may include a characters remaining field 1201 for informing the user how many text characters may be added to an in-process message. The media center controller 300 may determine the number of characters remaining based on the display characteristics and capabilities of the receiving wireless device as maintained using a database.

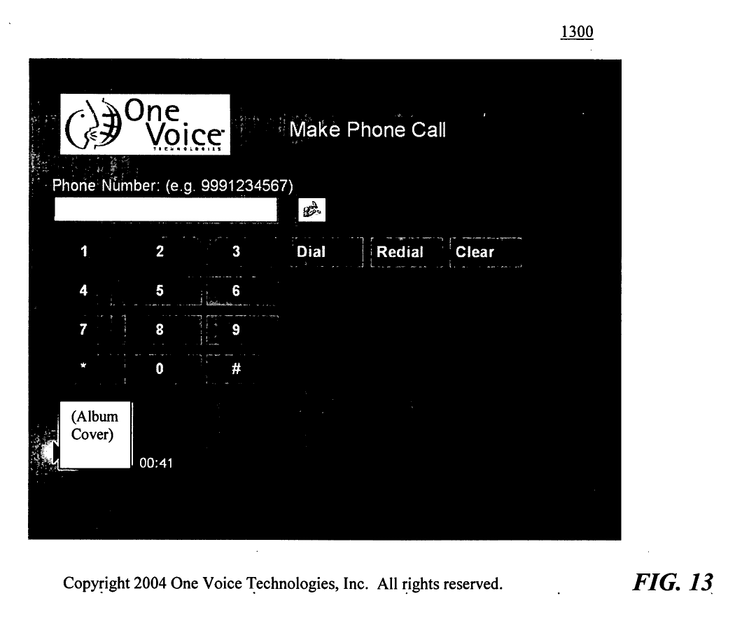

Furthermore, FIG. 13 shows a voice activated dialing interactive page 1300 according to at least one embodiment. Referring to FIG. 13, the voice activated dialing interactive page 1300 may provide an interface by which a user may make voice-activated telephone calls by speaking a name, nickname, or number. Users can store all of their contact information using a user account interactive page, such as shown in FIG. 15, of the media center controller 300. In at least one embodiment, there is no need to train the media center controller 300 to recognize each name.

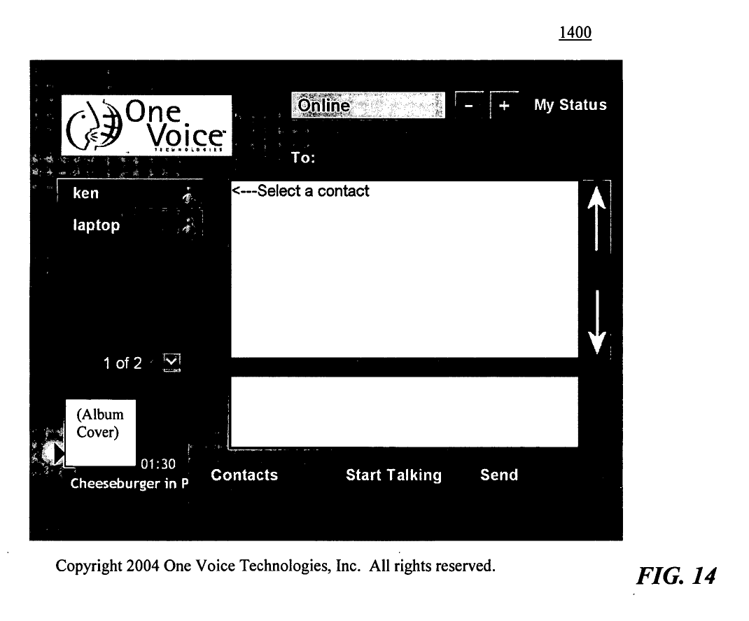

FIG. 14 shows a Windows Messenger™ interactive page 1400 according to at least one embodiment. Referring to FIG. 14, the Windows Messenger™ interactive page 1400 may provide an interface by which a user may communicate in real-time with other people who use Windows Messenger™ and who are signed in to the same instant messaging service. The media center controller 300 may allow users to send instant messages to each other by typing; to communicate through a PC-to-PC audio connection; or to communicate through a PC-to-PC audio/video connection.

In addition, the media center controller 300 may provide an interface by which a user may access voice mail systems (VM) by voice command over the telephone network. In particular, upon a (spoken or keyboard/keypad entered) command from the user to connect to his VM, the media center controller will connect to VM by dialing, connecting the call, and automatically playing the proper VM Connect tone to the far-end VM system (for example, a “*” tone), and then automatically (if so selected by the user) playing the VM user account number and password, as appropriate, through DTMF.

In an embodiment, this automated activity may be transparent to the user. After the user states “[name] Voice Mail,” the user hears Music On Hold (MOH), or feedback to the user alerting him to wait for computer processing, until the request is recognized and the media center controller 300 has forwarded the account and password tones to the VM system. Next, the media center controller 300 may play a VM greeting from the VM system. When the connection to the VM system is complete (if the user provided an incorrect account number or password), the media center controller 300 may connect through anyway and the user will hear the VM system request proper authorization keys. At this point, the media center controller 300 will have connected the VM outgoing line to the user so he can hear the prompts, but the line from the user will be connected to the media center controller for voice recognition. If the user hits one or more DTMF keys, the DTMF tones may be passed through to the VM system. Note that a ‘##’ key sequence will still disconnect from the VM system (assuming that VM systems will not use ‘##’ for any commands).

Voice access to carrier voice mail, corporate voice mail, and personal voice mail (home answering machines) may all be provided in much the same manner.

In an embodiment, media center controller 300 voicemail may provide most-often-used features such as, but not limited to: Play Voice Mail; Playback/Rewind/Repeat; Pause; Fast Forward n secs, Fast Rewind n secs; Get Next/Skip Ahead; Get Previous; Delete/Erase; Save Voice Mail; Call Sender; Help/VM Menu. The system may also respond to requests such as “Help”, Tutorial,” and “All Options.” System response to such user requests will be analogous to how the system responds to these commands in other VUI sequences. Note that some VM systems do not support all of the features listed. Unsupported features may be removed from the media center controller 300 prompts and online help, or the media center controller 300 will play a prompt to indicate that the requested feature is not supported by the active VM system. A simple command (e.g., “[Get my | Call my] Voice Mail”) may connect to a caller's VM. If the user commands “VoiceMail” from the main menu, and the user has more than one VM setup, then the system may prompt “Say ‘Verizon’ or ‘One Voice’ or ‘Home.’” From the main menu, for multiple VMs (e.g., carrier and corporate) the caller may be able to select which VM system he wants: “Voice Mail for Verizon”, “Verizon VoiceMail”, or “Voice Mail for One Voice.”

In an embodiment, the user may set up for multiple VM systems, choosing from carrier, business and home VM, by interacting with VM systems externally to them and using their own commands. The fields defining a VoiceMail entry may include: friendly name, provider selection list box, password required checkbox, and password text field that is masked for security. If the ‘Provider’ selection is “Other”, then other fields including a selection identifying the VM Connect key sequence (usually ‘#’) may be displayed and need to be entered. In an embodiment, many VM systems appear in the dropdown listbox for ‘Provider’ to make the selection easier for the user. The selections may include the a) carrier name(s), b) corporate VM systems, and c) identifiers for particular answering machines. Clear identification of the VM system may need to also identify the VM by product name, model number or version number. Knowing the type of VM service allows the media center controller 300 to automate the call setup sequence. If the user chooses “Other”, then details such as ‘VM Connect’ sequence, key mapping and timing requirements must be entered by the user. An example of this concern involves entry of the ‘VM Connect’ sequence, followed by the password. Some VM systems allow ‘#12345’ (VM Connect=‘#’, password=‘12345’) to be entered as one sequence. Other systems require a delay between ‘#’ and ‘12345.’

The following example describes how a user of the media center controller 300 may access carrier voicemail. First, from the main menu, the user may say, “Voice Mail.” The media center controller 300 may respond with, for example, “Just a moment while I connect you to [your voice mail system].” The media center controller 300 may then call the VM system. Upon connect, the media center controller 300 may issue a “VM Connect” DTMF (‘#’ for Service Provider 1, ‘*’ for Service Providers 2 and 3), if required, n msecs after off-hook and then DTMF the user's account number and/or password n msecs after it DTMFed the “VM Connect”. If the account number or password retrieved from the data store is bad, the media center controller 300 may not know that and it will still connect, but the login to the VM system will then fail. If the VM system hangs up, the media center controller 300 may respond with, for example, “Sorry, we could not connect to [ . . . ].”

For connection to the VM, the user must have entered their VM account and/or password on the media center controller 300 interactive page. The ‘Voice Mail Account Number’ field for the carrier may be visible only if the user has Voice VM service provided by the carrier. The ‘Voice Mail Password’ field for the carrier may be visible only if the user has Voice VM service provided by the carrier. For corporate or home VM access, the password field is always visible.

Furthermore, in at least one embodiment, the media center controller 300 may include calendar management. Regarding calendar management, the media center controller 300 may allow a user to access calendar functions by speaking, “Calendar.” The media center controller 300 may respond with, for example, “OK. To access calendar features, say Add an appointment, Add a meeting request, Edit, Delete or Look up.” <3 second delay> “For a list of all options say All Options. You can also say Help or Tutorial.” In an embodiment, calendar main menu commands may include: Add [an] appointment; Add [a] meeting; Edit; Delete; Look up; [Main Menu, All Options, Help, Cancel, Tutorial]—these are available at most response points. Also, in the following scenarios the “Undo” command always takes the user back to the previous step.

For example, to add an appointment, the user may speak, “Add an appointment.” The media center controller 300 may respond with, for example, “OK. Please say the month and date of your appointment.” <3 second delay> “You can also say today, tomorrow, or a day of the week.” The user may reply, “October 20^{th}.” The media center controller 300 may respond, “Monday, October 20^{th}. At what time?” (The media center controller 300 may say the day, month and date followed by the year if the appointment occurs in the next year.) The user may reply with one of: “10 am to 11 am,” “10 o'clock,” “10 am for 2 hours,” “10 am,” or “All day.” The media center controller 300 may respond with, for example, “October 20^{th}, 10 am to 11 am. What is the subject of your appointment?” The user may reply, “Doctor's appointment.” The media center controller 300 may save as a .wav file as an attachment or link, as with VR, and then say, “Please say the location.” To which the user may reply, “Scripps Clinic.” The media center controller 300 may save as a .wav file as an attachment or link, as with VR. Variations of this scenario as possible. For example, the media center controller 300 may allow the user to “look up” his calendar for a given day or period and, by interacting with the media center controller 300, receive his calendar schedule for that period. For example, the media center controller 300 may say, “MV: You have <#> appointment(s) today, October 21^{st}. First appointment is <appointment>. Second appointment is <appointment>.” Further, the user will have the option to choose where he/she would like the calendar alerts sent (e.g., mobile phone, e-mail at work, e-mail at home) under the preferences section of the user accounts interactive page of FIG. 15. In an embodiment, the Outlook™ default will be used to determine when the alert is sent out. Visual indications for calendar alerts may also be provided.

FIG. 15 shows a user account interactive page 1500 according to at least one embodiment. Referring to FIG. 15, the user account interactive page 1500 may provide an interface by which a user may create a profile with his preferences. To create a new user profile, users will click on the New User button 1501. They will be asked to provide their first and last name, greeting (how they want Media center controller to greet them at start up), e-mail address, and voice model (male or female). On this page, they will also have the option to choose IM setup, phone setup, e-mail setup, preferences, training, save, delete, or cancel.

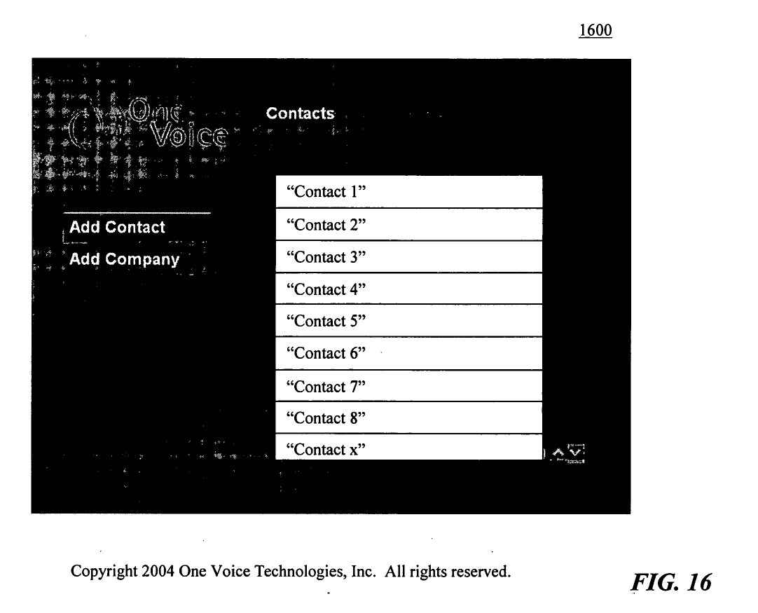

Furthermore, FIG. 16 shows a user contacts interactive page 1600 according to at least one embodiment. Referring to FIG. 16, the user contacts interactive page 1600 may provide an interface by which users may access all of their contacts from any controlled device that can access the media center. The media center communicator 300 may provide users voice access to all their important contact names and phone numbers so they don't have to carry an address book or PDA. Users can also add or edit contact information via voice input. In at least one embodiment, each of the FIGS. 8-16 may include certain interactive display items in addition to those described above beneficial to a user of a media center. For example, FIGS. 9 and 12-16 show an “album cover” icon in the lower left corner indicating the artist, album, song track, and length of play time remaining for an audio music selection.

Thus, the media center controller 300 may support a variety of media center functions and applications. Further details regarding the ability of the media center controller 300 to support bidirectional VOIP, PC-to-phone, and PC-to-PC communication are set forth below.

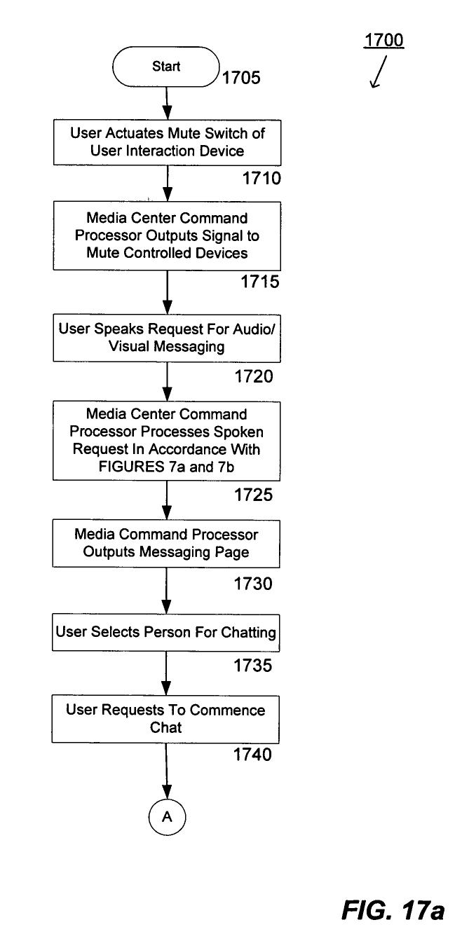

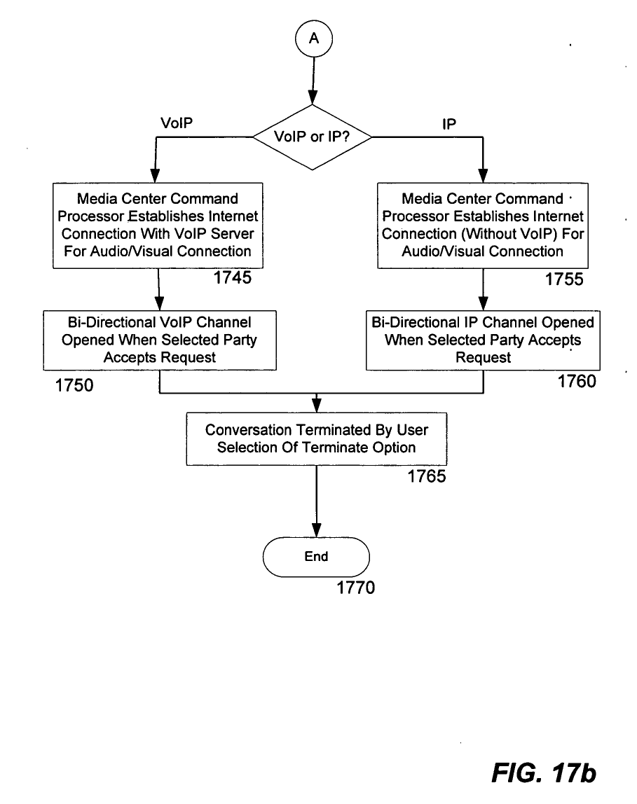

In an embodiment, the media center controller 300 may use a voice command capability to initiate PC-to-PC communications such as, for example, an Internet Messaging (IM) session, or VOIP communications. FIGS. 17a and 17b are a flowchart of a method 1700 for VoIP or PC-to-PC applications using the media center controller 300. Referring to FIG. 17a, a method 1700 may commence at 1705. Control may then proceed to 1710, while the top level menu (see, for example, FIG. 8) is displayed, the user may actuate mute switch on a user interaction device (for example, user interaction device 101).

Control may then proceed to 1715, at which in response to receiving a signal from the user interaction device that the mute switch has been actuated, the media center command processor may output a signal(s) to one or more controlled devices to mute the audio from the controlled devices.

Control may then proceed to 1720, at which the user may speak a request for an audio or audio/video messaging session. In an embodiment, the spoken request may be received by the user interaction device and provided therefrom to the media center command processor as described herein. Control may then proceed to 1725, at which the media center command processor may process the spoken request as set forth in FIGS. 7a and 7b herein.

Control may then proceed to 1730, at which a messaging interactive page may be displayed (see, for example, FIG. 1400). Control may then proceed to 1735, at which the user may select, via spoken request or manual selection, the person he wants to chat, in accordance with the processing described with respect to FIGS. 7a and 7b herein. Control may then proceed to 1740, at which the user may select, via spoken request or manual selection, to commence the chat session (e.g., selects the “Start Talking” option), in accordance with the processing described with respect to FIGS. 7a and 7b herein.

Control may then proceed to 1745 of FIG. 17b, at which the media center command processor may establish an Internet connection with a VOIP communication server to request an audio or audio/visual connection to the selected party. Control may then proceed to 1750, at which if the selected party accepts the request for a conversation, a bi-directional VOIP channel may be opened between the media center command processor and the user and the called party. A conversation may then ensue.

Alternatively, from 1740 control may then proceed to 1755 of FIG. 17b at which the media center command processor may establish an Internet connection with another computing device such as, for example, a PC, to request an audio or audio/visual connection to the selected party. Control may then proceed to 1760, at which if the selected party accepts the request for a conversation, a bi-directional IP channel may be opened between the media center command processor and the user and the called party.

Control may then proceed to 1765, at which the conversation may be terminated by the called party, or by the media center command processor user through selection, via spoken request or manual selection, of a terminate conversation option via, for example, the messaging screen (see, for example, FIG. 14), in accordance with the processing described with respect to FIGS. 7a and 7b herein. Control may then proceed to 1770, at which a method may end.

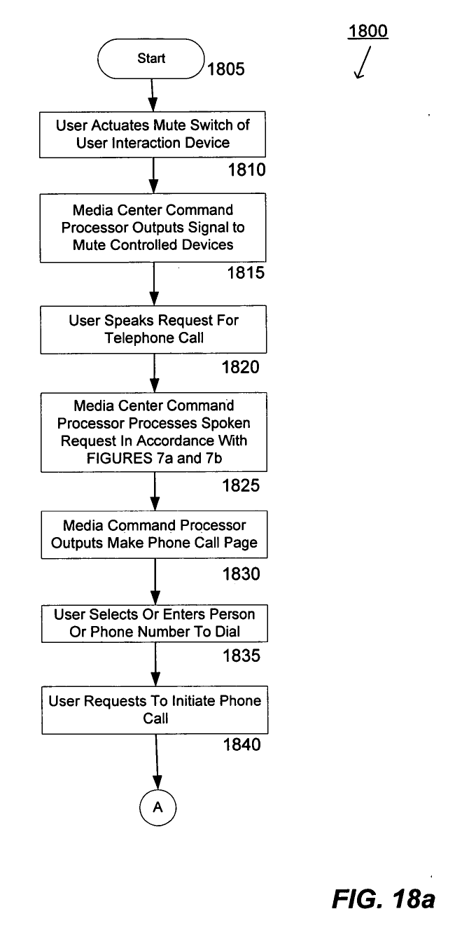

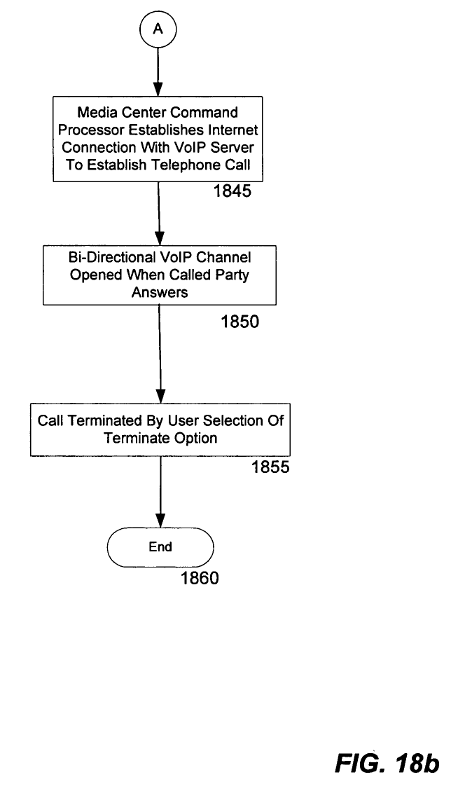

In an embodiment, the media center controller 300 may use a voice command capability to initiate PC-to-phone communications. FIGS. 18a and 18b are a flowchart of a method 1800 for PC-to-phone applications using the media center controller 300. Referring to FIG. 18a, a method 1800 may commence at 1805. Control may then proceed to 1810, while the top level menu (see, for example, FIG. 8) is displayed, the user may actuate mute switch on a user interaction device (for example, user interaction device 101).

Control may then proceed to 1815, at which in response to receiving a signal from the user interaction device that the mute switch has been actuated, the media center command processor may output a signal(s) to one or more controlled devices to mute the audio from the controlled devices.

Control may then proceed to 1820, at which the user may speak a request to make a telephone call. In an embodiment, the spoken request may be received by the user interaction device and provided therefrom to the media center command processor as described herein. Control may then proceed to 1825, at which the media center command processor may process the spoken request as set forth in FIGS. 7a and 7b herein.

Control may then proceed to 1830, at which a make phone call interactive page may be displayed (see, for example, FIG. 1300). Control may then proceed to 1835, at which the user may select, via spoken request or manual selection, the person he wants to chat or the telephone to which he wants to connect, in accordance with the processing described with respect to FIGS. 7a and 7b herein. Control may then proceed to 1840, at which the user may select, via spoken request or manual selection, to commence the initiate the telephone call (e.g., selects the “Dial” option), in accordance with the processing described with respect to FIGS. 7a and 7b herein.

Control may then proceed to 1845 of FIG. 18b, at which the media center command processor may establish an Internet connection with a VOIP communication server to request the telephone call to the selected party. Control may then proceed to 1850, at which if the selected party answers the incoming call, a request for a conversation, a bi-directional voice communication channel may be opened between the media center command processor and the user and the called party. In an embodiment, the called party may be accessed via the PSTN. In another embodiment, the called party may be accessed via an IP enabled phone, handset or communication device. In either case, the media center command processor may communicate with the called party using VOIP via VOIP gateway for conversion between IP and PSTN traffic. Optionally, the PSTN may also be used for voice connections with non-VOIP enabled called parties. A conversation may then ensue.

Control may then proceed to 1855, at which the call may be terminated by the called party, or by the media center controller user through selection, via spoken request or manual selection, of a terminate call option via, for example, the make phone call interactive page (such as, for example, FIG. 13), in accordance with the processing described with respect to FIGS. 7a and 7b herein. Control may then proceed to 1860, at which a method may end.

Thus has been shown a media center controller that includes a computing device having a user dialog manager to process commands and input for controlling one or more controlled devices of a media center. The system and methods may include the capability to receive and respond to commands and input from a variety of sources, including spoken commands from a user, for remotely controlling one or more electronic devices. The system and methods may also include a user interaction device capable of receiving spoken user input and transferring the spoken input to the computing device.

While the invention has been described with reference to the certain illustrated embodiments, the words that have been used herein are words of description, rather than words of limitation. Changes may be made, within the purview of the associated claims, without departing from the scope and spirit of the invention in its aspects. Although the invention has been described herein with reference to particular structures, acts, and materials, the invention is not to be limited to the particulars disclosed, but rather can be embodied in a wide variety of forms, some of which may be quite different from those of the disclosed embodiments, and extends to all equivalent structures, acts, and, materials, such as are within the scope of the associated claims.

1. A media center controller system comprising:
a computing device having at least one interface to one or more controlled devices; and
a media center command processor coupled to the computing device, the media center command processor including an interface to a handheld device, wherein the media center command processor includes a user dialog manager, a data/command processor, and a sequence processor;
wherein the media center command processor is configured to receive audio input from a handheld device and to perform, in response to the input received from the handheld device, at least one of: speech recognition processing, voice over Internet Protocol communications, instant messaging, electronic mail messaging, and control of one or more controlled devices.

2. The media center controller system of claim 1, wherein the media center command processor is further configured to receive manual input from the handheld device.

3. The media center controller system of claim 1, wherein the media center command processor further comprises:
a speech recognition processor; and
an audio feedback generator;
wherein the sequence processor is configured to process grammar or sequence data;
wherein the user dialog manager is configured to transfer an audio signal to the speech recognition processor, to receive audio feedback from the audio feedback generator, to transfer non-spoken input to the data/command processor, and to receive sequence information from the sequence processor;
wherein the computing device is configured to output interpreted command information to the one or more controlled devices, to output video information to a display monitor based on input received by the user dialog manager, and to output audio feedback to a user.

4. The media center controller system of claim 1, further comprising:
a handheld user interaction device configured to receive input from a user and including an interface to the media center command processor for transferring user input to the media center command processor.

5. The media center controller system of claim 4, wherein the computing device is configured to output audio feedback information and remote control commands received from the media center command processor to the user interaction device, and wherein the user interaction device is configured to output remote control commands to the one or more controlled devices.

6. The media center controller system of claim 5, wherein the user interaction device is configured to output audio feedback to a user.

7. The media center controller system of claim 4, wherein the computing device is configured to output audio feedback information to at least one controlled device.

8. The media center controller system of claim 4, wherein the computing device is configured to output video information to a display monitor.

9. The media center controller system of claim 4, in which the input received from a user includes audio input.

10. The media center controller system of claim 9, in which the input received from a user includes keypad input.

11. The media center controller system of claim 10, in which the input received from a user includes touchscreen input.

12. The media center controller system of claim 4, wherein the user interaction device is a remote control unit further including a microphone, and wherein the remote control unit is configured to transmit the audio signal to the computing device.

13. The media center controller system of claim 4, wherein the user interaction device is configured to receive audio feedback information and remote control commands from the computing device.

14. The media center controller system of claim 13, wherein the remote control unit includes a speaker.

15. The media center controller system of claim 12, in which the remote control unit further includes a mute switch, the remote control unit being configured to send a mute signal to the controlled devices through the computing device upon actuation of the mute switch and to send an unmute signal to the controlled devices through the computing device upon release of the mute switch.

16. The media center controller system of claim 15, in which the remote control unit controls the computing device.

17. The media center controller system of claim 1, in which the media center command processor is included in the computing device.

18. The media center controller system of claim 3, in which the speech recognition processor further includes a natural language processor configured to interpret spoken commands.

19. The media center controller system of claim 3, in which the audio signal represents speech provided by a user.

20. The media center controller system of claim 3, in which the audio signal is received via voice over Internet Protocol.

21. The media center controller system of claim 1, further comprising one or more controlled devices configured to output audio to a user using a speaker in response to receiving audio feedback information from the computing device.

22. The media center controller system of claim 1, in which the media center command processor is a headend system.

23. A method comprising:
receiving user input;
transferring the received user input for interpretation;
classifying the user input as audio input or non-spoken input;
transferring an audio signal to a speech recognition processor for interpretation of the audio signal into command or data information;
transferring non-spoken information to a data/command processor for validation;
providing, by the speech recognition processor or data/command processor, an indication of the interpreted command(s) or input;
transferring the interpreted command(s) or input to a sequence processor for validation;
obtaining sequence steps;
identifying valid commands at each sequence step;
transitioning from step to step within a sequence or between sequences;
validating the interpreted command or input to be within an acceptable range and received in sequence for an associated task as specified in a predefined state table;
preparing audio feedback to the user action;
preparing, using a visual output formatter, a visual response to the input; and
outputting the response to the user.

24. The method of claim 23, in which the audio input is received from a remote control device.

25. The method of claim 23, in which the non-spoken input is received via manual data entry source.

26. The method of claim 23, in which the audio input is received via voice over Internet Protocol.

27. The method of claim 23, in which the audio input is received public switched telephone network.

28. The method of claim 23, further comprising outputting the audio response to one or more controlled devices configured to output the audio response to a user using a speaker.

29. The method of claim 23, further comprising performing natural language processing to interpret the audio signal containing ambiguities.

30. The method of claim 23, further comprising obtaining command set and sequence information associated with the user input from grammar/sequence data.

31. The method of claim 30, in which the state table is contained in the grammar/sequence data.

32. The method of claim 23, further comprising:
sending a mute signal to the controlled devices during user speech input; and
sending an unmute signal to the controlled devices following user speech input.

33. A remote control device comprising:
a microphone for receiving spoken user input; and
a first interface to a computing device, wherein the first interface may further include an audio receiver portion for receiving audio from the computing device, an audio transmitter portion for providing an audio signal to the computing device, and a function key transmitter portion for transferring keypad information to the computing device.

34. The remote control device of claim 33, further comprising command keys.

35. The remote control device of claim 34, in which the command keys include a numeric keypad, a clear button, an enter button, and navigation buttons for up, down, left, right movement.

36. The remote control device of claim 33, further comprising a speaker for outputting audio to a user.

37. The remote control device of claim 33, further comprising a second interface to at least one controlled device.

38. The remote control unit of claim 33, in which the remote control unit controls the computing device.

39. The remote control unit of claim 33, in which the remote control unit includes an interface to a headend system.

40. A media center controller system comprising:
a computing device including an application processor and a media center command processor, wherein the media center command processor includes a user dialog manager;
a handheld user interaction device coupled to the computing device;
wherein the user dialog manager further includes a speech recognition processor, an audio feedback generator including a speech synthesizer, a data/command processor, and a sequence processor;
wherein the speech recognition processor is configured to generate a text output converted from spoken utterances, the speech recognition processor further including a natural language processor;
wherein the user dialog manager is configured to transfer an audio signal to the speech recognition processor, to receive synthesized speech from the speech synthesizer from the audio feedback generator, to receive pre-recorded audio files from the audio feedback generator for audio feedback to a user, to transfer non-spoken input to the data/command processor, and to receive sequence information from the sequence processor;
the sequence processor being coupled to a grammar/sequence database;
a speech synthesizing processor for generating a synthesized speech output in response to text data;
an interface to one or more controlled devices;
wherein the computing device is configured to output synthesized speech and pre-recorded audio information and remote control commands to the user interaction device and to output interpreted command information to at least one controlled device and video information to a display monitor, based on input received by the user dialog manager;
wherein the user interaction device coupled to the computing device and is configured to receive audio input from a user, the user interaction device further including an interface to the computing device for transferring user input to the computing device and a remote control interface to one or more controlled devices, and the user interaction device further configured to output remote control commands to the one or more controlled devices and to output synthesized speech or pre-recorded audio;
wherein the user interaction device further includes: a microphone and a speaker, and wherein the remote control unit is configured to transmit the audio signal to the computing device and to receive synthesized speech information, pre-recorded audio, and remote control commands from the computing device, and wherein the remote control unit further includes a mute switch, the remote control unit being configured to send a mute signal to the controlled devices through the media center command processor upon actuation of the mute switch and to send an unmute signal to the controlled devices through the media center command processor upon release of the mute switch;
an audio input system for receiving speech input provided by the user;
a video input system for receiving a live camera feed;
an audio output system for outputting synthesized speech to the user;
a keyboard entry system for input of user commands;
a display device for outputting visual responses and interactive pages to the user;
wherein the user dialog manager is logically connected through operating system services to input/output devices, the audio input system, the audio output system, the speech recognition processor and the speech synthesizing processor, and other computer-internal components;
a data set for storing and accessing user-related information, such as user profiles, contact information, and selected preferences; and
a data store for recorded audio or audio/visual files.

41. The media center controller system of claim 40, in which the controlled devices include a radio receiver for playing radio stations requested by the user.

42. The media center controller system of claim 40, in which the controlled devices include a television receiver for playing or recording television programs.

43. The media center controller system of claim 40, in which the controlled devices include an audio file/track player to play audio files requested by the user.

44. The media center controller system of claim 40, in which the controlled devices include an audio/visual player to play audio/visual files or tracks requested by the user.

45. The media center controller system of claim 40, in which the audio signal represents speech provided by a user.

46. The media center controller system of claim 40, in which the non-spoken input is received via manual data entry source.

47. The media center controller system of claim 40, in which the audio signal is received via voice over Internet Protocol.

48. The media center controller system of claim 40, in which the audio signal is received via public switched telephone network.

49. The media center controller system of claim 40, further comprising one or more controlled devices configured to output audio to a user using a speaker in response to receiving audio information from the computing device.

50. The media center controller system of claim 40, in which the media center command processor is a headend system.

51. A computer readable medium upon which is embodied a sequence of instructions which, when executed by a processor, cause the processor to be configured to:
receive user input;
transfer the received user input for interpretation;
classify the user input as audio input or non-spoken input;
transfer an audio signal to a speech recognition processor for interpretation of the audio signal into command or data information;
transfer non-spoken information to a data/command processor for validation;
provide, by the speech recognition processor or data/command processor, an indication of the interpreted command(s) or input;
transfer the interpreted command(s) or input to a sequence processor for validation;
validate the interpreted command or input to be within an acceptable range and received in sequence for an associated task as specified in a predefined state table;
prepare, using a speech synthesizer or a pre-recorded audio file, an audio response to the input;
prepare, using a visual output formatter, a visual response to the input; and
output the response to the user.

52. The computer readable medium of claim 51, in which the audio input is received from a remote control device.

53. The computer readable medium of claim 51, in which the non-spoken input is received via manual data entry source.

54. The computer readable medium of claim 51, in which the audio input is received via voice over Internet Protocol.

55. The computer readable medium of claim 51, in which the audio input is received via public switched telephone network.

56. The computer readable medium of claim 51, further comprising outputting the audio response to one or more controlled devices configured to output the audio response to a user using a speaker.

57. The computer readable medium of claim 51, further comprising performing natural language processing to interpret the audio signal containing ambiguities.

58. The computer readable medium of claim 51, further comprising obtaining command set and sequence information associated with the user input from grammar/sequence data.

59. The computer readable medium of claim 51, in which the state table is contained in the grammar/sequence data.

60. The computer readable medium of claim 51, further comprising outputting the audio response to a user via a speaker of the controlled device.

61. The computer readable medium of claim 51, further comprising:
sending a mute signal to the controlled devices during user speech input; and
sending an unmute signal to the controlled devices following user speech input.

62. A method comprising:
sending a mute signal one or more controlled devices upon user actuation of a mute switch on a user interaction device;
receiving spoken user input in which the user input includes a request for audio or visual messaging;
transferring the received user input for interpretation;
classifying the user input as audio input;
transferring an audio signal to a speech recognition processor for interpretation of the audio signal into command or data information;
providing, by the speech recognition processor or data/command processor, an indication of the interpreted command(s) or input;
transferring the interpreted command(s) or input to a sequence processor for validation;
obtaining sequence steps;
identifying valid commands at each sequence step;
transitioning from step to step within a sequence or between sequences;
validating the interpreted command or input to be within an acceptable range and received in sequence for an associated task as specified in a predefined state table;
preparing audio feedback for an audio response to the user action;
preparing, using a visual output formatter, a messaging page;
outputting the response to the user;
selecting a person for messaging;
establishing an Internet connection and opening a bi-directional channel therein; and
terminating the messaging session.

63. The method of claim 62, in which the bi-directional channel is a voice over Internet Protocol channel.

64. A method comprising:
sending a mute signal one or more controlled devices upon user actuation of a mute switch on a user interaction device;
receiving spoken user input in which the user input includes a request to make a telephone call;
transferring the received user input for interpretation;
classifying the user input as audio input;
transferring an audio signal to a speech recognition processor for interpretation of the audio signal into command or data information;
providing, by the speech recognition processor or data/command processor, an indication of the interpreted command(s) or input;
transferring the interpreted command(s) or input to a sequence processor for validation;
obtaining sequence steps;
identifying valid commands at each sequence step;
transitioning from step to step within a sequence or between sequences;
validating the interpreted command or input to be within an acceptable range and received in sequence for an associated task as specified in a predefined state table;
preparing, using a speech synthesizer or a pre-recorded file for playback, an audio response to the input;
preparing, using a visual output formatter, a make telephone call page;
outputting the response to the user;
selecting a person or telephone number for a telephone call;
establishing an Internet connection with a voice over Internet Protocol server and opening a bi-directional voice over Internet Protocol channel therein; and
terminating the telephone call.
